(19) United States
(12) Patent Application Publication
(10) Pub. No.: US 2008/0071353 A1
(43) Pub. Date: Mar. 20, 2008

(54) ENDOPROSTHESIS CONTAINING MAGNETIC INDUCTION PARTICLES

(75) Inventors: Jan Weber, Maastricht (NL); Liliana Atanasoska, Edina, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/855,693

(22) Filed: Sep. 14, 2007

Related U.S. Application Data
(60) Provisional application No. 60/845,136, filed on Sep. 15, 2006.

(51) Int. Cl. A61F 2/06

(52) U.S. Cl. 623/1.15

(57) ABSTRACT

Endoprostheses (e.g., stents) containing one or more magnetic induction particles (e.g., nanoparticles) are disclosed.

### CROSS-REFERENCE TO RELATED APPLICATIONS

This application claims priority under 35 USC §119(e) to U.S. Provisional Patent Application Ser. No. 60/845,136, filed on Sep. 15, 2006, the entire contents of which are hereby incorporated by reference.

### TECHNICAL FIELD

This invention relates to medical devices, such as endoprostheses, and methods of making and using the same.

### BACKGROUND

The body includes various passageways including blood vessels such as arteries, and other body lumens. These passageways sometimes become occluded or weakened. For example, they can be occluded by a tumor, restricted by plaque, or weakened by an aneurysm. When this occurs, the passageway can be reopened or reinforced, or even replaced, with a medical endoprosthesis. An endoprosthesis is an artificial implant that is typically placed in a passageway or lumen in the body. Many endoprostheses are tubular members, examples of which include stents, stent-grafts, and covered stents.

Many endoprostheses can be delivered inside the body by a catheter. Typically the catheter supports a reduced-size or compacted form of the endoprosthesis as it is transported to a desired site in the body, for example the site of weakening or occlusion in a body lumen. Upon reaching the desired site the endoprosthesis is installed so that it can contact the walls of the lumen.

One method of installation involves expanding the endoprosthesis. The expansion mechanism used to install the endoprosthesis may include forcing it to expand radially. For example, the expansion can be achieved with a catheter that carries a balloon in conjunction with a balloon-expandable endoprosthesis reduced in size relative to its final form in the body The balloon is inflated to deform and/or expand the endoprosthesis in order to fix it at a predetermined position in contact with the lumen wall. The balloon can then be deflated, and the catheter withdrawn.

When the endoprosthesis is advanced through the body, its progress can be monitored, e.g., tracked, so that the endoprosthesis can be delivered properly to a target site. After the endoprosthesis is delivered to the target site, the endoprosthesis can be monitored to determine whether it has been placed properly and/or is functioning properly. Methods of tracking and monitoring a medical device include X-ray fluoroscopy and magnetic resonance imaging (MRI). MRI is a non-invasive technique that uses a magnetic field and radio waves to image the body. In some MRI procedures, the patient is exposed to a magnetic field, which interacts with certain atoms, e.g., hydrogen atoms, in the patient's body. Incident radio waves are then directed at the patient. The incident radio waves interact with atoms in the patient's body, and produce characteristic return radio waves. The return radio waves are detected by a scanner and processed by a computer to generate an image of the body.

### SUMMARY

In one aspect, the invention features an endoprosthesis, e.g., a stent, that includes a bioerodible portion and a plurality of magnetic induction particles, the particles having a metal coating.

In another aspect, the invention features an endoprosthesis, e.g., a stent (e.g., a drug delivering stent) having a substantially tubular polymer body and that includes magnetic induction particles having a size of about 1 to 1000 nm.

In yet another aspect, the invention features a method of implanting an endoprosthesis (e.g., stent) in a body passageway of an organism and applying a magnetic field to the endoprosthesis to control one or more of the erosion rate of the erodible portion, and/or the permeability of the stent to body fluid. The method includes visualizing the stent by MRI or X-ray fluoroscopy.

In yet another aspect, the invention features a method of making an endoprosthesis (e.g., stent) that includes providing a plurality of metal particles, said particles having a size of about 1 to 500 nm, and a functionalized organic surface; forming a dispersion of magnetic particles in a polymer, and utilizing said dispersion to form an endoprosthesis (e.g., stent).

Embodiments may include one or more of the following features. The magnetic particles are typically ferromagnetic or super-paramagnetic. The magnetic particles contain a metal chosen from one or more of iron, nickel or cobalt. The magnetic particles can be coated with a radiopaque material. The magnetic particles are coated with a metal, e.g., gold, platinum or silver. The magnetic particles can be chosen from one or more of: Co@Au, Co@Ag, Fe3O4@Au, Fe3O4@Ag, FePt and/or CoFe@Au. The magnetic particles have a diameter from about 10 to 1000 nm, more typically, about 3 to 50 nm. The magnetic particles have a volume from about 10 to 500 cubic nm. The magnetic particles include a polymer coating or a polyelectrolyte coating. The magnetic particles can be coupled to one or more functional group chosen from, e.g., an alkyl, di- or tri-fluoromethyl, hydroxyl, ether, carboxylic acid, ester, amide, halogen (e.g., chloro, bromo), nitrile, amine, borate, alkene, alkyne, diacetylene, aryl, oligo(phenylene ethylene), quinone, oligo(ethylene glycol), sulfone, epoxide, pyrene, azobenzene, silyl, carbonyl, imide, anhydride, thiol, ammonium, isocyanate or urethane.

Embodiments may also include one or more of the following features. The magnetic particles are bonded to, or embedded within, the erodible portion. The magnetic particles are in a separate layer from the erodible portion. The erodible portion is the polymer body. The magnetic particles are located within one or more of: a polyelectrolyte coating, a conducting polymer, an amphiphylic block copolymer, and/or within an inorganic coating (e.g., a silica coating). The magnetic particles are attached to a surface of the stent, e.g., the particles are covalently bound to the stent.

Further embodiments may also include one or more of the following features. The endoprosthesis, e.g., stent, can further include a therapeutic agent or drug. The therapeutic agent can be embedded in the bioerodible portion or contained in a capsule. The therapeutic agent can be chosen from, e.g., one or more of: an anti-thrombogenic agent, an anti-proliferative/anti-mitotic agents, an inhibitor of smooth muscle cell proliferation, an antioxidant, an anti-inflammatory agent, an anesthetic agents, an anti-coagulant, an antibiotic, and an agent that stimulates endothelial cell growth and/or attachment. In one embodiment, the therapeutic agent is paclitaxel. The magnetic particles are embedded in a common layer with the drug. The common layer can be bioerodible (e.g., a bioerodible metal (e.g., magnesium or iron), a bioerodible metal alloy, a bioerodible polymer, or a mixture thereof ) or non-bioerodible. The common layer is a polymer. The drug is in a coating on the stent, e.g., a bioerodible or non-bioerodible coating on the stent.

Other embodiments may include one or more of the following: Forming a dispersion by combining said particles and polymer in an organic solvent; incorporating a drug into said polymer; combining said drug with said particles in said dispersion; and/or applying said dispersion to a stent body.

An erodible or bioerodible medical device, e.g., a stent, refers to a device, or a portion thereof, that exhibits substantial mass or density reduction or chemical transformation, after it is introduced into a patient, e.g., a human patient. Mass reduction can occur by, e.g., dissolution of the material that forms the device and/or fragmenting of the device. Chemical transformation can include oxidation/reduction, hydrolysis, substitution, electrochemical reactions, addition reactions, or other chemical reactions of the material from which the device, or a portion thereof, is made. The erosion can be the result of a chemical and/or biological interaction of the device with the body environment, e.g., the body itself or body fluids, into which it is implanted and/or erosion can be triggered by applying a triggering influence, such as a chemical reactant or energy to the device, e.g., to increase a reaction rate. For example, a device, or a portion thereof, can be formed from an active metal, e.g., Mg or Ca or an alloy thereof, and which can erode by reaction with water, producing the corresponding metal oxide and hydrogen gas (a redox reaction). For example, a device, or a portion thereof, can be formed from an erodible or bioerodible polymer, or an alloy or blend erodible or bioerodible polymers which can erode by hydrolysis with water. The erosion occurs to a desirable extent in a time frame that can provide a therapeutic benefit. For example, in embodiments, the device exhibits substantial mass reduction after a period of time which a function of the device, such as support of the lumen wall or drug delivery is no longer needed or desirable. In particular embodiments, the device exhibits a mass reduction of about 10 percent or more, e.g. about 50 percent or more, after a period of implantation of one day or more, e.g. about 60 days or more, about 180 days or more, about 600 days or more, or 1000 days or less. In embodiments, the device exhibits fragmentation by erosion processes. The fragmentation occurs as, e.g., some regions of the device erode more rapidly than other regions. The faster eroding regions become weakened by more quickly eroding through the body of the endoprosthesis and fragment from the slower eroding regions. The faster eroding and slower eroding regions may be random or predefined. For example, faster eroding regions may be predefined by treating the regions to enhance chemical reactivity of the regions. Alternatively, regions may be treated to reduce erosion rates, e.g., by using coatings. In embodiments, only portions of the device exhibits erodibilty. For example, an exterior layer or coating may be erodible, while an interior layer or body is non-erodible. In embodiments, the endoprosthesis is formed from an erodible material dispersed within a non-erodible material such that after erosion, the device has increased porosity by erosion of the erodible material.

Erosion rates can be measured with a test device suspended in a stream of Ringer's solution flowing at a rate of 0.2 m/second. During testing, all surfaces of the test device can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

Aspects and/or embodiments may have one or more of the following additional advantages. The endoprosthesis, e.g., stent, can include particles, e.g., nanoparticles, having ferromagnetic or super-paramagnetic properties, e.g., the particles that contain, e.g., a ferromagnetic metal, such as cobalt or iron, or a mixture thereof. Such particles can be coated with a surface (e.g., a gold- or silver-surface) that increases their compatibility with stent coatings, their stability, reduces their toxicity in vivo, and/or facilitates attachment of one or more functional groups. The rate of erosion and/or biodegradation of different portions of the endoprostheses can be controlled. For example, erosion (e.g., biocrosion) of selected areas of, or the entire, endoprosthesis can be accelerated using non-invasive means (e.g., by applying a magnetic field). The endoprostheses may not need to be removed from a lumen after implantation. The porosity of an endoprosthesis, e.g., a drug eluting stent, can be controlled, e.g., increased, by embedding and, optionally removing, the magnetic particles. Release of a therapeutic agent from an endoprosthesis, e.g., a polyclectrolyte coated stent, can be controlled using non-invasive means (e.g., a magnetic field). The visibility of the endoprosthesis, e.g., biodegradable endoprosthesis, to imaging methods, e.g., X-ray and/or Magnetic Resonance Imaging (MRI), can be enhanced, even after the endoprosthesis is partly eroded. Furthermore, attachment of different functional groups to the surface of the particles increases the number of applications where the endoprosthesis can be used.

Other aspects, features, and advantages will be apparent from the description and drawings, and from the claims.

### DESCRIPTION OF DRAWINGS

FIG. 1 is a perspective view of a stent.

FIG. 2 is a cross-sectional view of a stent wall.

FIG. 3 is a cross-sectional view of a magnetic induction particle having an outer and an inner portion.

FIGS. 4A-4D are longitudinal cross-sectional views, illustrating delivery of a stent in a collapsed state (FIG. 4A), expansion of the stent (FIG. 4B) and deployment of the stent (FIG. 4C). FIG. 4D depicts degradation in the presence of a magnetic field.

FIGS. 5A-5B are cross-sectional views of a stent having a base surrounded by a multiple layers, in the absence and presence of a magnetic field, respectively.

FIG. 6 is a partial cross-section of a stent having capsules attached to its surface.

FIG. 7 is a cross-sectional view of a capsule.

Like reference symbols in the various drawings indicate like elements.

### DETAILED DESCRIPTION

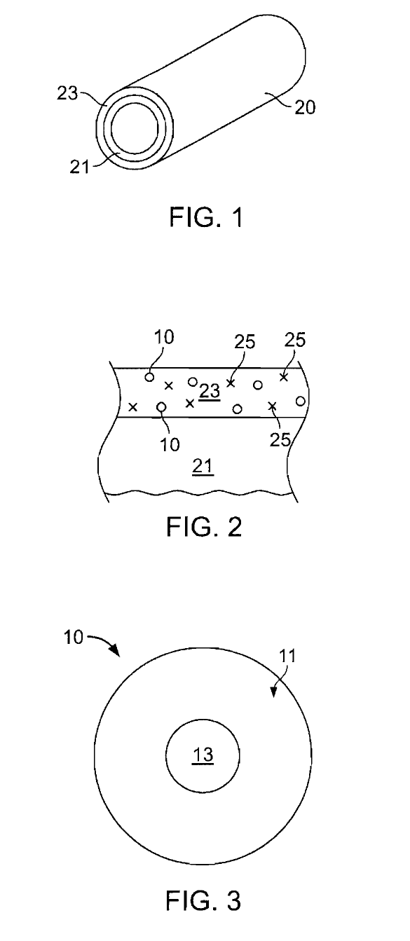

Referring to FIG. 1, a stent 20 is a generally tubular device adapted for use in a body lumen. Referring as well to FIG. 2, a cross-section through the stent wall, the stent includes a first layer 21 and a second layer 23. The first layer 21 is a bioerodible material, e.g. a polymer or a metal. The second layer 23 incorporates a therapeutic agent 25 and plurality of magnetic induction particles 10, which when exposed to a magnetic field are agitated. Referring to FIG. 3, a cross-section through a single particle, the particles 10 are preferably multilayer nanoparticles including an inner core 13 of magnetic induction material and an outer coating 11 of a metal or nonmetal. The magnetic induction material is contained within the particles, e.g., nanoparticles, which in turn may be coated with one or more layers to, e.g., increase biocompatibility, increase radiopacity, among others.

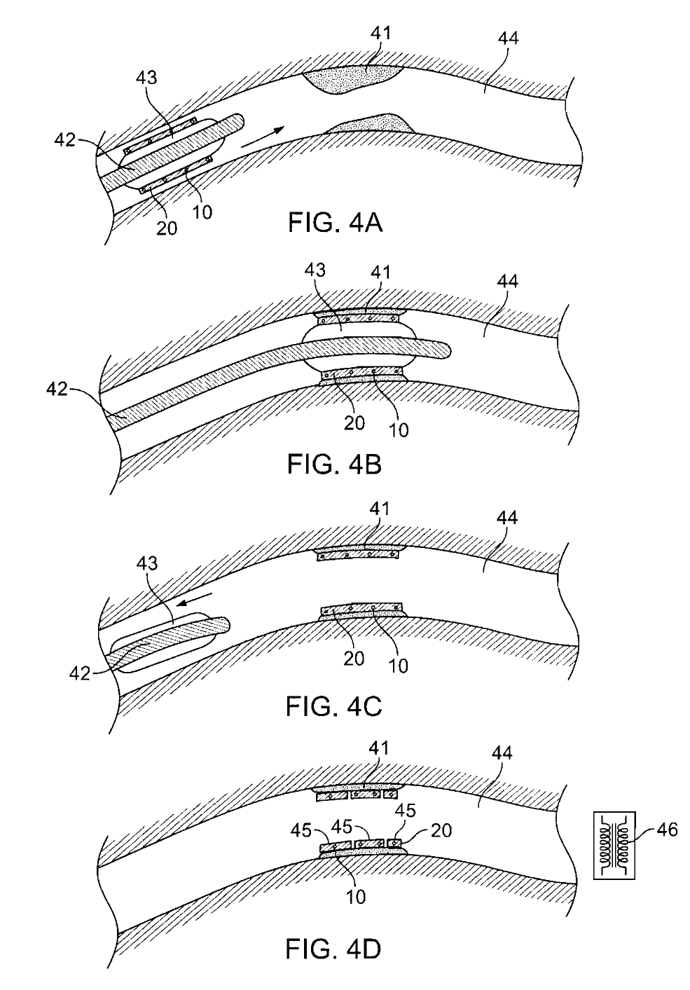

Referring as well to FIGS. 4A-4D, in use stent 20 is placed over a balloon 43 carried near the distal end of a catheter 42, and is directed through a lumen 44 (FIG. 4A) until the portion carrying the balloon and stent reaches the region of an occlusion 41. The stent 20 is then radially expanded by inflating the balloon 43 and pressed against the vessel wall with the result that occlusion 41 is compressed, and the vessel wall surrounding it undergoes a radial expansion (FIG. 4B). The pressure is then released from the balloon and the catheter 42 is withdrawn from the vessel (FIG. 4C). Referring to FIG. 4D, the stent 20 is exposed to a magnetic field 46 (e.g., an alternating field), which causes agitation of the induction particles and/or displacement of the induction particles inside the coating. The agitation of the induction particles may increase the porosity and/or erosion rate of the stent into fragments 45. In one embodiment, the agitation enhances the permeability of the second layer 23 to body fluid which facilitates release of the therapeutic agent 25 and/or accelerates erosion of the first layer 21. The magnetic field can be selectively applied, e.g. by positioning a patient in a MRI machine or positioning a field generator close to the stent from outside the body or inside the body, e.g. using a catheter. The field strength and duration can be applied selectively to selectively accelerate erosion of the stent and/or elution of the drug. For example, applying a strong field from an MRI machine can dislodge the induction particles from the coating, or even completely remove them out of the coating leaving behind a porous structure. As another example, a Neodynium magnet can be mounted on a guide wire and spun at high speed within a thin catheter tube located inside the stent. The spinning inside of the catheter prevents damage to the vessel wall. Such magnets are commercially available from Micro-Magnet Technology Co., Ltd, China); for example, a rotor magnet for quartz watch stepping motor made out of SmCo5 or Sm2Co17, and having a size of OD0.8˜1.6 mm±0.005 Diameter of hole: 0.2˜0.6 mm±0.01, and a height: 0.3˜1.0 mm±0.01 can be used.

The size of the particles and their composition facilitate incorporation of the particles in the stent and can enhance one or more of: erosion rate, drug delivery and/or radiopacity, of the stent. In embodiments, the induction particles are nanoparticles. The nanoparticles can have at least one dimension (e.g., the thickness for a nanoplate, the diameter for a nanosphere, a nanocylinder and a nanotube) that is less than 1000 nm, e.g., less than 100 nm. In particular embodiments, the magnetic particles have a spherical shape with a diameter ranging from about 1 nm to 100 nm; more typically, from about 1 nm to 50 nm; from about 3 nm to 25 nm; from about 5 to 15 nm; or about 10 nm. In certain embodiments, the magnetic particles of the endoprosthesis have a diameter larger, or smaller, than 10 nm.

In embodiments, the particles, e.g., nanoparticles, of the endoprosthesis have an inner portion 13 that is ferromagnetic, paramagnetic or super-paramagnetic. For example, the particles can have an inner portion that includes a ferromagnetic metal, a paramagnetic metal, or a mixture thereof. Particles containing ferromagnetic metals may show ferromagnetic or super-paramagnetic properties depending on their size. For example, particles having a diameter larger than 10 nm can show ferromagnetic properties at and above room temperature, whereas particles below 10 nm show super-paramagnetic properties. Exemplary ferromagnetic metals include iron, nickel and cobalt, or a mixture thereof A particular particle is gold-coated cobalt spherical nanocrystals in a size range of 5-25 nm. Exemplary paramagnetic metals that can be used in the inner portion of the magnetic particles include magnesium, molybdenium, lithium and tantalum. Magnetic particles are further discussed in Bao, Y. et al. (2005) Journal of Magnetism and Magnetic Materials 293:15-19. In one embodiment, ferromagnetic FeCo particles are used (Hutten, A. et al. (2005) Journal of Magnetism and Magnetic Materials 293:93-101). Such particles typically range in size from about 1 to 11 nm and are superparamagnetic.

The particles typically also include an outer portion made up of one or a plurality of layers that can enhance dispersibility in a stent layer, enhance radiopacity, increase stability of the inner portion (e.g., increased corrosion protection), reduce toxicity in an organism by reducing exposure to less compatible metal particles (e.g., cobalt particles) and/or facilitates attachment of one or more functional groups or layers. In one embodiment, the outer portion includes a radiopaque, biocompatible metal, such as gold and silver, which encapsulates less biocompatible materials, e.g. Co. Exemplary magnetic particles contained in the endoprosthesis, e.g., stent, include gold-coated cobalt particles (Co@Au), silver-coated cobalt particles (Co@Ag), gold-coated magnetic iron oxide (Fe₃O₄@Au), silver-coated magnetic iron oxide (Fe₃O₄@Ag) and gold-coated cobalt/iron mixtures (CoFe@Au), iron platinum alloys (FePt), or a combination thereof. Gold- or silver-coated cobalt particles (Co@Au or Co@Ag) are typically used. Fabrication of Co@Au particles is described in Lu et al. (2005) Langmuir 21(5):2042-50. Magnetite containing magnetic particles having a gold or a silver shell are discussed in Madhuri, M. et al. (2005) Journal of Colloidal and Interface Science 286:187-194. Radiopaque metals are described in Heath U.S. Pat. No. 5,725,570.

In embodiments, the outer portion of the particle includes a polymer or another organic material. The organic material may be provided directly over a core or the material may be provided over an intermediate layer, e.g. a metal layer such as a radiopaque layer, over the core. In embodiments, the particles can be derivatized, e.g., coupled (e.g., covalently coupled) to one or more functional moieties. In some embodiments, a metal outer portion or surface of the magnetic particle is treated with an agent that adds one or more thiol groups forming, e.g., thiocarbamate or dithiocarbamate ligands. In one embodiment, a gold metal surface can be treated by chemisorption of thiols or carbodithioate (—CS₂) to attach one or more thiol end groups. For example, dithiocarbamate ligands 1-11 on a gold surface are readily formed by immersing a gold substrate in solutions with an equimolar ratio of carbon disulfide (CS₂) and a secondary amine. Suitable thiol groups are discussed in H. Schmidbaur, Gold-Progress in Chemistry, Biochemistry and Technology, Wiley, New York 1999; Zhao, Y. et al. (2005) J. Am. Chem. Soc. 127:7328-7329. In one embodiment, the particles are capped or coated with tetra-benzylthiol groups and carbonylic acids to enhance dispersibility in solvents such as toluene. Such capping will facilitate direct mixing of the particles with organic polymers and solvents, such as styrene-isobutylene-styrene (SIBs) and biodegradable polyamide-polyester based drug eluting coatings and organic solvents, such as toluene. Coating of particles is described further in Balasubramanian, R. et al. (2002) Langmuir 18:3676-3681.

The outer portion of the magnetic particles can also include one or more functional groups chosen from, e.g., an alkyl, di- or tri-fluoromethyl, hydroxyl, ether, carboxylic acid, ester, amide, halogen (e.g., chloro, bromo), nitrile, amine, borate, alkene, alkyne, diacetylene, aryl, oligo(phenylene ethylene), quinone, oligo(ethylene glycol), sulfone, epoxide, pyrene, silyl, carbonyl, imide, anhydride, thiol, ammonium, isocyanate, urethane, or azobenzene. A Table describing some examples of functional groups that have been incorporated into self-assembled monolayer whether within the interior of the film or at the terminus is set forth at page 7 of Smith, R. K. et al. (2003) Progress in Surface Science 75:1-68. Additional examples of surface modification of the magnetic particles include modification of gamma-Fe₂O₃ nanoparticles with aminopropylsilyl (APS) groups in 3-aminopropyltriethoxysilane (Iida, H. et al. (2005) Electrochimica Acta 51:855-859); ozone modification of a lyophobic surface of the magnetic particles capped with oleic acid to form carbonyl and carboxyl groups (Lee, S. et al. (2006) Journal of Colloid and Interface Science 293:401-408); and modification of the surface of magnetite particles with an amine or an amino surface (Shieh, D-B. et al. (2005) Biomaterials 26:7183-7191, Ashtari, P. et al. (2005) Talanta 67:548-554). In embodiments, a functional group bound to a gold or silver surface of a particle is coupled (e.g., covalently coupled) to a polymer in which the particle is embedded, e.g. a biocrodible polymer. A particle can be attached to each polymer chain to facilitate a homogenous distribution of the particles in the polymer. The outer portion of the magnetic particle can be a protein, polynucleotide or other biomolecules. In embodiments, the particles include polyelectrolyte coatings. Polyelectrolytes are polymers having charged (e.g., ionically dissociable) groups. The number of these groups in the polyelectrolytes can be so large that the polymers are soluble in polar solvents (including water) when in ionically dissociated form (also called polyions). Depending on the type of dissociable groups, polyelectrolytes can be classified as polyacids and polybases. When dissociated, polyacids form polyanions, with protons being split off. Polyacids include inorganic, organic and biopolymers. Examples of polyacids are polyphosphoric acids, polyvinylsulfuric acids, polyvinylsulfonic acids, polyvinylphosphonic acids and polyacrylic acids. Examples of the corresponding salts, which are called polysalts, are polyphosphates, polyvinyl sulfates, polyvinylsulfonates, polyvinylphosphonates and polyacrylates. Polybases contain groups that are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations. Some polyelectrolytes have both anionic and cationic groups, but nonetheless have a net positive or negative charge.

The polyelectrolytes can include those based on biopolymers. Examples include alginic acid, gum arabicum, nucleic acids, pectins and proteins, chemically modified biopolymers such as carboxymethyl cellulose and lignin sulfonates, and synthetic polymers such as polymethacrylic acid, polyvinylsulfonic acid, polyvinylphosphonic acid and polyethylenimine. Linear or branched polyelectrolytes can be used. Using branched polyelectrolytes can lead to less compact polyelectrolyte multilayers having a higher degree of wall porosity. In some embodiments, polyelectrolyte molecules can be crosslinked within or/and between the individual layers, to enhance stability, e.g., by crosslinking amino groups with aldehydes. Furthermore, amphiphilic polyelectrolytes, e.g., amphiphilic block or random copolymers having partial polyelectrolyte character, can be used in some embodiments to affect permeability towards polar small molecules.

Other examples of polyelectrolytes include low-molecular weight polyclectrolytes (e.g., polyelectrolytes having molecular weights of a few hundred Daltons up to macromolecular polyclectrolytes (e.g., polyelectrolytes of synthetic or biological origin, which commonly have molecular weights of several million Daltons). Still other examples of polyelectrolyte cations (polycations) include protamine sulfate polycations, poly(allylamine) polycations (e.g., poly(allylamine hydrochloride) (PAH)), polydiallyldimethylammonium polycations, polyethyleneimine polycations, chitosan polycations, gelatin polycations, spermidine polycations and albumin polycations. Examples of polyelectrolyte anions (polyanions) include poly(styrenesulfonate) polyanions (e.g., poly(sodium styrene sulfonate) (PSS)), polyacrylic acid polyanions, sodium alginate polyanions, eudragit polyanions, gelatin polyanions, hyaluronic acid polyanions, carrageenan polyanions, chondroitin sulfate polyanions, and carboxymethylcellulose polyanions. In embodiments, the particles do not include an outer portion, rather the particles consist of inductive material, e.g. of nanometer dimensions.

Referring back to FIG. 2, the cross-section through the stent wall, in embodiments, the particles are embedded in a separate layer 23 over an erodible material 21. The layer 23 can be provided only on the outside of the stent as illustrated. Alternatively or in addition, the layer 23 can be provided on the inside of the stent. The layer 23 can be formed of an erodible material or non-erodible material. In embodiments, the layer is a drug-eluting coating, such as a polymer, e.g., styrene-isobutylene-styrene (SIBs). In embodiments, the layer 23 has a thickness of about 0.5 to 20 micrometer. The layer 21 has a thickness of about 1 to 300, typically about 10 to 200 micrometer. In embodiments, induction particles and/or drug are provided in the layer 21, as well as or in addition to the layer 23. The particles, when agitated, can enhance the permeability of layers adjacent to the layers in which they are incorporated. In embodiments, the particles are agitated sufficiently to heat the layer they are incorporated in and/or adjacent layers. In other embodiments, the stent has a single layer forming the stent wall, which includes induction particles and optionally drug.

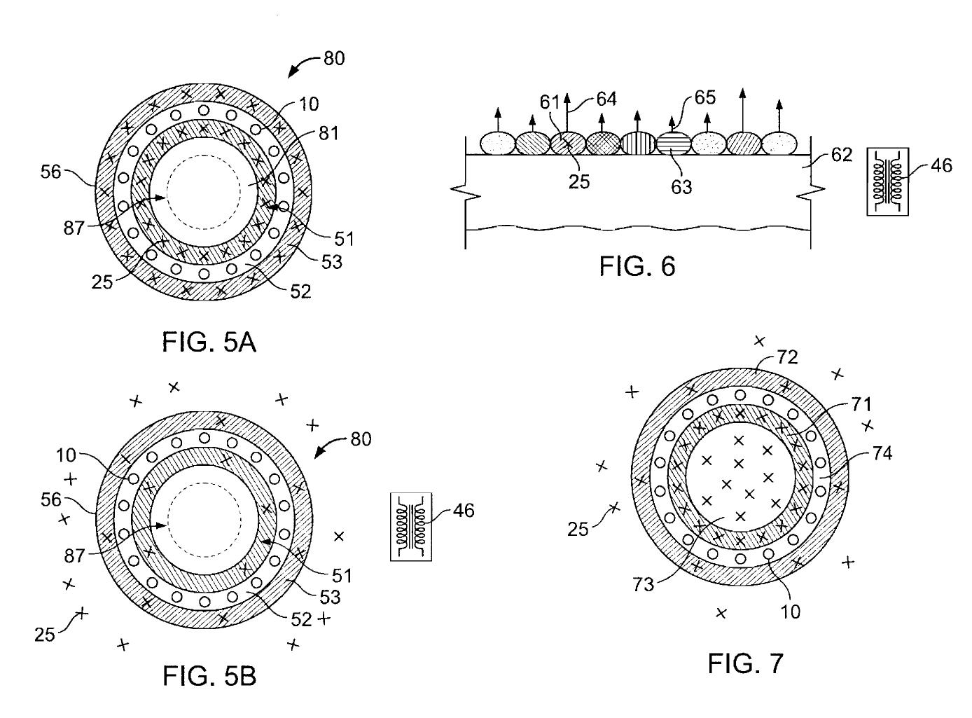

Referring to FIGS. 5A-5B, cross-sectional views of an embodiment of a stent 80 having at least four layers are shown in the absence and presence of a magnetic field 46, respectively. The stent 80 has a base 87 surrounded by a layer 51 containing a therapeutic agent 25; a layer 52 including one or more magnetic induction particles, and, optionally, one or more layers, exemplified herein as layer 53, optionally, containing the same or a different therapeutic agent 25 or a radiopaque material (e.g., pure gold nanoparticles) (see FIG. 5A). Referring to FIG. 5B, applying a rapidly oscillating magnetic field 46 causes agitation of the magnetic particles, increasing the permeability of the layers 51, 52, 53 which enhances elution of the therapeutic agent. In a particular embodiment, one or more of layers 51, 52, 53 include polyelectrolytes and the magnetic particles may be provided in a uniform layer surrounding the stent body. For example, since the gold or silver surfaces of the magnetic particles, e.g., Co@Au, are typically positively charged at neutral pH, these surfaces can be coated with a negatively charged layer of, e.g., anionic polyclectrolytes. One or more charged layers, e.g., alternating cationic and anionic polyelectrolyte layers, can be sequentially coated onto the layer containing the magnetic particles. One or more therapeutic agents and/or radiopaque material can be disposed on or within the multi-layered structure.

In particular embodiments, ferromagnetic cobalt nanoparticles are coated with gold shells and embedded into polyelectrolyte capsules fabricated with layer-by-layer assembly of poly(sodium) 4-styrene sulfonate) and poly(allylamine hydrochloride). Application of low frequency alternating magnetic fields (1200 Oe strength, 100-300 Hz) to such magnetic capsules increases in their wall permeability. Multilayer polyelectrolyte structures are described in Lu et al. (2005) supra. The base 87 can be a non-erodible material, e.g., a polymer or a metal (e.g. stainless steel) or an erodible material (such as a polymer or metal). In particular embodiments, the base is an erodible metal such as magnesium or iron. Application of a magnetic field can enhance erosion by increasing permeability of the layers 51, 52, 53.

In certain embodiments, a charged therapeutic agent is used, and one or more layers of the charged therapeutic agent are deposited during the course of assembling multi-layer structure 56. For example, the therapeutic agent can be a polyelectrolyte (e.g., where the therapeutic agent is a polypeptide or a polynucleotide) and it is used to create one or more polyelectrolyte layers within multi-layer structure 56. In other embodiments, the charged therapeutic agent is not a polyelectrolyte (e.g., it may be a charged small molecule drug), but one or more layers of the charged therapeutic agent can be substituted for one or more layers of the same charge (i.e., positive or negative) during the layer-by-layer assembly process. The therapeutic agent can be charged, for example, because it is itself a charged molecule or because it is intimately associated with a charged molecule. Examples of charged therapeutic agents include small molecule and polymeric therapeutic agents containing ionically dissociable groups. In embodiments in which the therapeutic agent does not possess one or more charged groups, it can nevertheless be provided with a charge, for example, through non-covalent association with a charged species. Examples of non-covalent associations include hydrogen bonding, and hydrophilic/lipophilic interactions. For instance, the therapeutic agent can be associated with an ionic amphiphilic substance.

Referring to FIG. 6, a stent 62 has on its surface a series of capsules 61 containing one or more therapeutic agents 25. Referring to FIG. 7, the therapeutic agent 25 is contained in a lumen 73 within the capsule and/or in one or more layers 71, 72, e.g., polymeric or polyelectrolyte layers, surrounding the capsule lumen 73. A layer of magnetic particles 74 surrounds the capsule lumen 73. In alternative embodiments, the magnetic particles are localized within the capsule, or dispersed within the capsule lumen itself. The capsules can be charged and can be formed, for example, using layer-by-layer techniques such as those described in commonly assigned U.S. Ser. No. 10/985,242, U.S. application publicly available through USPTO Public Pair, and U.S. Ser. No. 10/768,388, published as U.S. Ser. No. 05/0129727 by Weber, J and Robaina, S. In embodiments, one or more layers of the charged capsules can be deposited during the course of the layer-by-layer assembly process. In one embodiment, the capsules are attached to the surface of the endoprosthesis, e.g., stent, by ionic attraction. In embodiments, the capsules are attached by embedding them using, e.g., a polyelectrolite coating on the stent. The capsules can be made of a biodegradable material, e.g., have a biodegradable outer layer or shell. The outer layer can be chosen to be permeable to the therapeutic agent, e.g., a lipid or phospholipids layer. In some embodiments, the capsules are sized to facilitate absorption by the body over time. In one embodiment, the capsules include one or more therapeutic agents typically embedded within or in between one or more layers, e.g., a polymeric or polyelectrolyte layer, and a layer comprised of one or more magnetic particles. In certain embodiments, the capsule may differ from each other containing different layers, number of magnetic particles and/or therapeutic agents. In one embodiment, the capsules have a diameter of about 1μ to 300μ, e.g. about 50 to 100μ. The release of the therapeutic agent will depend on factors such as the therapeutic agent being released, the number of magnetic particles embedded in the polyelectrolyte layer, and the porosity of the polymer layer. For example, referring back to FIG. 6, a capsule 61 containing a higher number of magnetic particle particles will typically release a greater amount of a therapeutic agent 25 than the release 65 of a capsule 63 containing less particles, upon exposure to a magnetic field 46. In embodiments, multiple capsules with different drugs and/or release profiles (different pattern as in FIG. 6) are provided. The release of the drugs can be controlled sequentially by controlling the field strength and/or duration applied to the capsules.

In other embodiments, the particles can be used to form a porous coating in a stent, e.g., a drug eluting stent. For example, particles present in a polymer coating of a stent can be removed by applying, e.g., a magnetic field, a change in pH, heat or solvent (e.g., toluene), leaving a porous coating. The size of the pores can be adjusted by varying the diameter and/or the number of particles. For example, magnetic particles embedded in a weak polymer film (gel) can be displaced by applying a strong magnetic field, leaving behind vertical shafts in the polymer film. Spirals or other complex channels in the polymer film can be created by changing the direction of the magnetic field during the movement of the particles through the polymer film. Such alterations to the polymer film are typically made using soft gel like polymers, which can be crosslinked after the particles are removed. Alternatively, a polymer solution containing a plurality of magnetic particles embedded within or coated, e.g., in an outer coating can be applied, e.g., sprayed or dip coated, on a surface. The magnetic particles can be removed while the solvent is still evaporating from the coating. As yet another example, a porous coating can be created by embedding or coating a plurality of magnetic particles, e.g., FeCo nanoparticles (e.g., Fe₅₀Co₅₀), in a polymer film. Such FeCo nanoparticles typically range in size from about 1 to 11 nm, are typically superparamagnetic, and have a high magnetophoretic mobility (Hutten, A. et al. (2005) Journal of Magnetism and Magnetic Materials 293:93-101). Upon application of a magnetic field, the particles can be dislodged by magnetic attraction or agitation resulting in a porous coating. In other embodiments, a mesoporous carbon containing magnetic particles (e.g., iron oxide nanoparticles) embedded in the carbon walls can be synthesized as described in Lee, J. et al. (2005) Carbon 43:2536-2543. The approach described by Lee et a. (2005) supra can be extended to the synthesis of magnetically separable ordered mesoporous carbons containing various pore structures.

Suitable biocrodible materials include one or more of a metallic component (e.g., a metal or alloy), a non-metallic component (e.g., a biodegradable polymer), or any combination thereof. Biocrodible materials are described, for example, in U.S. Pat. No. 6,287,332 to Bolz; U.S. Patent Application Publication No. 2002/0004060 A1 to Heublein; U.S. Pat. Nos. 5,587,507 and 6,475,477 to Kohn et al. Examples of biocrodible metals include alkali metals, alkaline earth metals (e.g., magnesium), iron, zinc, and aluminum. Examples of bioerodible metal alloys include alkali metal alloys, alkaline earth metal alloys (e.g., magnesium alloys), iron alloys (e.g., alloys including iron and up to seven percent carbon), and zinc alloys. Examples of bioerodible non-metals include bioerodible polymers, such as, e.g., polyanhydrides, polyorthoesters, polylactides, polyglycolides, polysiloxanes, cellulose derivatives and blends or copolymers of any of these. Biocrodible polymers are disclosed in U.S. Published Patent Application No. 2005/0010275, filed Oct. 10, 2003; U.S. Published Patent Application No. 2005/0216074, filed Oct. 5, 2004; and U.S. Pat. No. 6,720,402.

In other embodiments, the stent can include one or more biostable materials in addition to one or more bioerodible materials. For example, the bioerodible material may be provided as a coating in a biostable stent body. Examples of biostable materials include stainless steel, tantalum, nickel-chrome, cobalt-chromium alloys such as Elgiloy® and Phynox®, Nitinol (e.g., 55% nickel, 45% titanium), and other alloys based on titanium, including nickel titanium alloys, thermo-memory alloy materials. Stents including biostable and biocrodible regions are described, for example, in U.S. patent application Ser. No. 11/004,009, filed on Dec. 3, 2004, and entitled “Medical Devices and Methods of Making the Same”. The material can be suitable for use in, for example, a balloon-expandable stent, a self-expandable stent, or a combination of both (see e.g., U.S. Pat. No. 5,366,504).

The stent can be manufactured, or the starting stent can be obtained commercially. Methods of making stents are described, for example, in U.S. Pat. No. 5,780,807 and U.S. Application Publication 2004/0000046-A1. Stents are also available, for example, from Boston Scientific Corporation, Natick, Mass., USA, and Maple Grove, Minn., USA. The stent can be formed of any biocompatible material, e.g., a metal or an alloy, as described herein. The biocompatible material can be suitable for use in a self-expandable stent, a balloon-expandable stent, or both. Examples of other materials that can be used for a balloon-expandable stent include noble metals, radiopaque materials, stainless steel, and alloys including stainless steel and one or more radiopaque materials.

Charged layers containing the polyelectrolytes can be assembled with layers containing magnetic particles using a layer-by-layer technique in which the layers electrostatically self-assemble. Methods for layer-by-layer assembly are disclosed in commonly assigned U.S. Ser. No. 10/985,242, U.S. application publicly available through USPTO Public Pair. For example, the layer-by-layer assembly can be conducted by exposing a selected charged substrate (e.g., stent) to solutions or suspensions that contain species of alternating net charge, including solutions or suspensions that contain charged magnetic particles, polyelectrolytes, and, optionally, charged therapeutic agents and/or other radiopaque nanoparticles. The concentration of the charged species within these solutions and suspensions, which can be dependent on the types of species being deposited, can range, for example, from about 0.01 mg/ml to about 30 mg/ml. The pH of these suspensions and solutions can be such that the magnetic clusters, polyclectrolytes, and optional therapeutic agents and/or nanoparticles maintain their charge. Buffer systems can be used to maintain charge. The solutions and suspensions containing the charged species (e.g., solutions/suspensions of magnetic clusters, polyclectrolytes, or other optional charged species such as charged therapeutic agents and/or charged nanoparticles) can be applied to the charged substrate surface using a variety of techniques. Examples of techniques include spraying techniques, dipping techniques, roll and brush coating techniques, techniques involving coating via mechanical suspension such as air suspension, ink jet techniques, spin coating techniques, web coating techniques and combinations of these processes. Layers can be applied over an underlying substrate by immersing the entire substrate (e.g., stent) into a solution or suspension containing the charged species, or by immersing half of the substrate into the solution or suspension, flipping the same, and immersing the other half of the substrate into the solution or suspension to complete the coating. In some embodiments, the substrate is rinsed after application of each charged species layer, for example, using a washing solution with a pH that maintains the charge of the outer layer.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

The endoprosthesis, e.g., the stent, can, further include at least one therapeutic agent chosen from one or more of, e.g., an anti-thrombogenic agent, an anti-proliferative/anti-mitotic agents, an inhibitor of smooth muscle cell proliferation, an antioxidant, an anti-inflammatory agent, an anesthetic agents, an anti-coagulant, an antibiotic, or an agent that stimulates endothelial cell growth and/or attachment. Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application No. 2005/019265A.

To enhance the radiopacity of stent 20, a radiopaque material, such as gold nanoparticles, can be incorporated into multi-layered structure 56. For example, gold nanoparticles can be made positively charged by applying a outer layer of lysine to the nanoparticles, e.g., as described in “DNA Mediated Electrostatic Assembly of Gold Nanoparticles into Linear Arrays by a Simple Dropcoating Procedure” Murali Sastrya and Ashavani Kumar, Applied Physics Letters, Vol. 78, No. 19, 7 May 2001. Other radiopaque materials include, for example, tantalum, platinum, palladium, tungsten, iridium, and their alloys. Radiopaque materials are also disclosed in Heath U.S. Pat. No. 5,725,570.

Medical devices, in particular endoprostheses, as described above include implantable or insertable medical devices, including catheters (for example, urinary catheters or vascular catheters such as balloon catheters), guide wires, balloons, filters (e.g., vena cava filters), stents of any desired shape and size (including coronary vascular stents, aortic stents, cerebral stents, urology stents such as urethral stents and ureteral stents, biliary stents, tracheal stents, gastrointestinal stents, peripheral vascular stents, neurology stents and esophageal stents), grafts such as stent grafts and vascular grafts, cerebral aneurysm filler coils (including GDC-Guglilmi detachable coils-and metal coils), filters, myocardial plugs, patches, pacemakers and pacemaker leads, heart valves, and biopsy devices. In one embodiment, the medical device includes a catheter having an expandable member, e.g., an inflatable balloon, at its distal end, and a stent or other endoprosthesis (e.g., an endoprosthesis or stent as described herein). The stent is typically an apertured tubular member (e.g., a substantially cylindrical uniform structure or a mesh) that can be assembled about the balloon. The stent typically has an initial diameter for delivery into the body that can be expanded to a larger diameter by inflating the balloon. The medical devices may further include drug delivery medical devices for systemic treatment, or for treatment of any mammalian tissue or organ.

The medical device, e.g., endoprosthesis, can be generally tubular in shape and can be a part of a stent. Simple tubular structures having a single tube, or with complex structures, such as branched tubular structures, can be used. Depending on specific application, stents can have a diameter of between, for example, 1 mm and 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stents can also be preferably bioerodible, such as a bioerodible abdominal aortic aneurysm (AAA) stent, or a bioerodiblc vessel graft.

In some embodiments, the medical device, e.g., endoprosthesis, is used to temporarily treat a subject without permanently remaining in the body of the subject. For example, in some embodiments, the medical device can be used for a certain period of time (e.g., to support a lumen of a subject), and then can disintegrate after that period of time. Subjects can be mammalian subjects, such as human subjects (e.g., an adult or a child). Non-limiting examples of tissues and organs for treatment include the heart, coronary or peripheral vascular system, lungs, trachea, esophagus, brain, liver, kidney, bladder, urethra and ureters, eye, intestines, stomach, colon, pancreas, ovary, prostate, gastrointestinal tract, biliary tract, urinary tract, skeletal muscle, smooth muscle, breast, cartilage, and bone.

All publications, patent applications, patents, and other references mentioned herein are incorporated by reference herein in their entirety.

Other embodiments are within the scope of the following claims.

What is claimed is:

1. A stent comprising a bioerodible portion and a plurality of magnetic induction particles, said particles having a metal coating.

2. The stent of claim 1, wherein the magnetic particles contain a metal selected from iron, nickel and cobalt.

3. The stent of claim 1, wherein the magnetic particles are coated with a radiopaque material.

4. The stent of claim 1, wherein the particles are coated with gold, platinum or silver.

5. The stent of claim 1, wherein the magnetic particles are selected from the group consisting of Co@Au, Co@Ag, Fe3O4@Au, Fe3O4®Ag, FePt and CoFe@Au.

6. The stent of claim 1, wherein the magnetic particles are ferromagnetic, paramagnetic or super-paramagnetic.

7. The stent of claim 1, wherein the magnetic particles have a diameter from about 10 to 1000 nm.

8. The stent of claim 1, wherein the particles have a diameter from about 3 to 50 nm.

9. The stent of claim 1, wherein the particles have a volume from about 10 to 500 cubic nm.

10. The stent of claim 1, wherein the particles include a polymer coating.

11. The stent of claim 1, wherein the magnetic particles are coupled to a functional group selected from the group consisting of an alkyl, di- or tri-fluoromethyl, hydroxyl, ether, carboxylic acid, ester, amide, halogen (e.g., chloro, bromo), nitrile, amine, borate, alkene, alkyne, diacetylene, aryl, oligo(phenylene ethylene), quinone, oligo(ethylene glycol), sulfone, epoxide, pyrene, azobenzene, silyl, carbonyl, imide, anhydride, thiol, ammonium, isocyanate and urethane.

12. The stent of claim 1, wherein the particles include a polyelectrolyte coating.

13. The stent of claim 1, wherein the particles are bonded to the erodible portion.

14. The stent of claim 1, wherein the particles are in a separate layer from the erodible portion.

15. The stent of claim 1, wherein the magnetic particles are embedded in the biocrodible portion.

16. The stent of claim 1, wherein the magnetic particles are located within a polyelectrolyte coating.

17. The stent of claim 1, wherein the magnetic particles are located within a conducting polymer.

18. The stent of claim 1, wherein the magnetic particles are located within an amphiphylic block copolymer.

19. The stent of claim 1, wherein the magnetic particles are located within a inorganic coating.

20. The stent of claim 1, wherein the particles are embedded in a common layer with a drug.

21. The stent of claim 20, wherein the common layer is a polymer.

22. The stent of claim 21, wherein the common layer is bioerodible.

23. The stent of claim 21, wherein the common layer is non-bioerodible.

24. The stent of claim 1, wherein the particles are attached to a surface of the stent.

25. The stent of claim 1, particles are covalently bound to the stent.

26. The stent of claim 1, wherein the bioerodible portion comprises a bioerodible metal, a bioerodible metal alloy, a bioerodible polymer, or a mixture thereof.

27. The stent of claim 26, wherein the bioerodible metal is magnesium or iron.

28. The stent of claim 1, further comprising at least one therapeutic agent.

29. The stent of claim 22, wherein at least one therapeutic agent is embedded in the bioerodible portion.

30. The stent of claim 28, wherein at least one therapeutic agent is contained in a capsule.

31. A stent comprising a substantially tubular polymer body and magnetic induction particles having a size of about 1 to 1000 nm.

32. The stent of claim 31 wherein the particles have a size of about 10 to 100 nm.

33. The stent of claim 31 wherein the particles are coated with a metal.

34. The stent of claim 31 wherein particles contain iron, nickel or cobalt and are coated with silver, gold or platinum.

35. The stent of claim 31 wherein the polymer body is bioerodible.

36. A drug delivering stent comprising a tubular body and including magnetic induction particles having a size of about 1 to 1000 nm.

37. The stent of claim 36, wherein the drug is in a coating on the stent.

38. The stent of claim 36, wherein the coating is bioerodible.

39. The stent of claim 36, wherein the coating is non-bioerodible.

40. The stent of claim 36, wherein the particles are in the coating.

41. A method comprising implanting the stent of claim 1 in a body passageway of an organism and applying a magnetic field to control erosion rate of the erodible portion.

42. The method of claim 41, comprising applying a magnetic field to control the permeability of the stent to body fluid.

43. The method of c]aim 41, comprising visualizing the stent by MRI or X-ray fluoroscopy.

44. A method of making a stent comprising:
providing a plurality of metal particles, said particles having a size of about 1 to 500 nm, and a functionalized organic surface
forming a dispersion of magnetic particles in a polymer, and
utilizing said dispersion to form a stent.

45. The method of claim 44 comprising forming said dispersion by combining said particles and polymer in an organic solvent.

46. The method of claims 44 or 45 comprising incorporating a drug into said polymer.

47. The method of claim 46 comprising combining said drug with said particles in said dispersion.

48. The method of claim 44 comprising applying said dispersion to a stent body.
